(19) United States
(12) Patent Application Publication
(10) Pub. No.: US 2022/0401118 A1
(43) Pub. Date: Dec. 22, 2022

(54) SYSTEMS AND METHODS FOR REMOVING MATERIAL FROM A VESSEL

(71) Applicant: STRYKER CORPORATION, Fremont, CA (US)

(72) Inventors: Roy Leguidleguid, Union City, CA (US); Michael P. Wallace, Pleasanton, CA (US); Robert J. Garabedian, Mountain View, CA (US); Jayson Delos Santos, Fremont, CA (US); Jason Iain Glithero, Oakland, CA (US)

(73) Assignee: STRYKER CORPORATION, Fremont, CA (US)

(21) Appl. No.: 17/890,607

(22) Filed: Aug. 18, 2022

Related U.S. Application Data
(63) Continuation of application No. PCT/US2020/018655, filed on Feb. 18, 2020.

(51) Int. Cl. A61B 17/221

(52) U.S. Cl. CPC A61B 17/221; A61M 25/0668

(57) ABSTRACT

Apparatus and methods employing same for loading and inserting an inverting tube apparatus into a body lumen, such as a blood vessel, including introducers for inverting tube apparatuses. Also described herein are stacked tractor regions and methods of using them for removing larger and/or longer materials from the body lumen. Also described herein are methods and apparatuses for assisting in the manual operation the inverting tube apparatuses described herein, including grips.

### CROSS-REFERENCE TO RELATED APPLICATIONS

This patent application is a continuation of International Application No. PCT/US2020/018655, filed Feb. 18, 2020, the entire disclosure of which is hereby incorporated by reference.

### INCORPORATION BY REFERENCE

All patent application publications and issued patents mentioned in this specification are herein incorporated by reference in their entirety to the same extent as if each individual publication or patent was specifically and individually stated to be incorporated by reference.

### FIELD

This apparatuses (e.g., systems) and methods described herein relate to mechanical removal of material from within a body lumen, such as, but not limited to, a blood vessel. For example, the methods and apparatuses described herein may be thrombectomy apparatuses and methods for removal of clot material from a blood vessel.

### BACKGROUND

It is often desirable to remove material, such as tissue from the body in as minimally invasive a manner as possible, so as not to damage other tissues. For example, removal of tissue from within a vasculature, such as blood clots, may improve patient conditions and quality of life.

Many vascular system problems stem from insufficient blood flow through blood vessels. One causes of insufficient or irregular blood flow is a blockage within a blood vessel referred to as a blood clot, or thrombus. Thrombi can occur for many reasons, including after a trauma such as surgery, or due to other causes. For example, a large percentage of the more than 1.2 million heart attacks in the United States are caused by blood clots (thrombi) which form within a coronary artery.

Clinical data indicates that clot removal may be beneficial or even necessary to improve patient outcomes. For example, in the peripheral vasculature, interventions and procedures can reduce the need for an amputation by 80 percent. The ultimate goal of any technique to treat these conditions is to remove the blockage and to restore patency, quickly, safely, and cost effectively. This has traditionally been achieved by thrombus dissolution, fragmentation, thrombus aspiration or a combination of these methods. More recently, devices for mechanically removing material, including thrombus material, from with a lumen of the vessel have been described.

Examples of devices that include an inverting tube for removing material from a body lumen, such as for removing a clot from a blood vessel (e.g., thrombectomy devices), are disclosed and described in each of U.S. Pat. No. 10,271,864, as well as in each of U.S. Patent Application Publication Nos. 2019/0117214, 2018/0042626 and 2018/0042624, and in U.S. patent application Ser. No. 16/566,393. These apparatuses do an excellent job at removing material from within a blood vessel, but in some situations may face challenges when using longer inverting tubes, and in particular, when using knitted inverting tubes that fit snugly over the inversion support catheter, for example, when constrained within a delivery catheter or in other low-profile situations. In some cases, the knitted tractor portion may jam or lock onto the outside of the inversion support catheter when removing material. This problem may be particularly acute when removing material from tortious vessels.

Thus, there is a need for devices, including thrombectomy devices, that can be remove tissue, and particularly large and/or hard materials, from within a body lumen without jamming or locking up. Described herein are apparatuses (devices, systems and kit) and methods of using them that may address the needs and problems discussed above.

### SUMMARY OF THE DISCLOSURE

The disclosed inventions relate to improvement in the operation, and in particular, the insertion and use of, inverting tube apparatuses for removing material from within a body. These apparatuses may generally include an inversion support, which may include a catheter and in some variations a funnel region at the distal end of the catheter, a flexible tube configured to move over the outside of the inversion support and invert into the inversion support, and in some variations a puller attached to a first end of the inversion support for pulling the flexible tube into the inversion support. These apparatuses may be generally referred to as apparatuses for removing a material from a vessel, and may be configured as mechanical thrombectomy apparatuses.

The methods and apparatuses described herein may provide improvements for introducing inverting tube apparatuses into the body, including in particular introduces for inserting inverting tube apparatuses including an expandable funnel at the distal end region of the apparatus. Also described herein are apparatuses configured to provide longer flexible tube regions that may assist in removing larger or longer amounts of material (e.g., clots), and methods of using them. Also described herein are system and methods that enhance the manual operation of these inverting tube apparatuses; and in particular, the operation of such apparatuses (e.g., systems) by a single operator using both hands. Also described herein are methods and apparatuses for managing long inversion support catheters and/or pullers. Any of these features, components and techniques may be used separately or in combination.

For example, described herein are apparatuses (e.g., systems) including one or more introducers for introducing an inverting tube apparatus into a sheath, or a hub of a sheath, so that they may be delivered into the body for deployment and operation. Any of these apparatuses may include an introducer that is configured to enable low-friction introduction of an apparatus including an expandable distal funnel (e.g., as part of the inversion support) into the body.

These apparatuses may be configured to be inserted into the body via a sheath so that the apparatus can be delivered, using a deployment cannula and/or guidewire, to the target region of the body. In variations in which the apparatus includes a funnel it may be otherwise difficult to transfer the expandable funnel portion of the apparatus into the sheath and therefore into the body. A standard introducer at the back of a sheath (e.g., sheath hub) may require large amounts of force in order to insert the portion of the apparatus including the expandable funnel; this force may result in partially or wholly collapsing and/or jamming the apparatus. For example, if the introducer is pushed in too hard or far into the sheath hub, the tip of the introducer may at least partially collapse circumferentially, pinching/grabbing the inversion support, e.g., at the funnel tip region, making it hard to transfer the inversion support into the sheath. Further, if the introducer is not pushed far enough into the sheath hub, this may result in a dead space that may make it hard to transfer the apparatus (e.g., the inversion support) into the sheath.

Described herein are apparatuses, introducer apparatuses that are configured to easily and cleanly collapse the expandable funnel portion of an inversion support and control the insertion depth of the introducer into the sheath hub. These introducer may also be configured as peel-away introducers and may include a stop element to guide how far the introducer may be inserted into sheath hub.

The introducers described herein may be configured as funnel introducers that are configured to allow a user to easily load the inverting tube apparatuses described herein (e.g., including an expandable funnel on the distal end region of the inversion support catheter) into the introducer prior to loading the apparatus into the sheath. In some variations these apparatuses may be used for re-loading an inversion support catheter (and flexible tube and puller) into the body, e.g., to remove additional material for subsequent treatments to the same patient at the same or different locations.

The introducers described herein are configured to allow an untrained user to correctly insert and position the apparatus into a sheath hub, e.g., with minimal manipulation (e.g., twisting) of the inversion support, and particularly the expandable funnel portion of the inversion support. These introducers (e.g., funnel introducers) may allow loading inverting tube apparatuses having inversion support catheters with expandable funnel. These methods and apparatuses may be used with any collapsible funnel (e.g., in particular, with any catheter including an expandable funnel).

Also described herein introducers configured to allow reloading of a second tractor (e.g., flexible tractor tube) into an inversion support catheter, or vice versa.

For example, described herein are devices (e.g., introducer devices, including funnel introducers) for introducing an inversion support catheter into a delivery sheath, the device comprising: an elongate body having a lumen extending from a distal end to a proximal end, the body configured to tear along a defined tear line extending proximally to distally, wherein the lumen has a narrower diameter on at a proximal end region of the body than at a distal end region of the body; a first flat region formed on a side of the body between the proximal end region and the distal end region and extending into the lumen; and a second flat region formed on the side of the body between the proximal end region and the distal end region and extending into the lumen.

A device for introducing an inversion support catheter into a delivery sheath (e.g., an introducer), as described herein may include: an elongate body having a lumen extending from a distal end to a proximal end, the body configured to tear along a defined tear line extending proximally to distally, wherein the lumen has a narrower inner diameter on at a proximal end region of the body than at a distal end region of the body; a stop projecting from an outer surface of the distal end region of the body, wherein the stop is configured to limit the depth that the device is insertable into the sheath; a first flat region formed onto a side of the body at a first location between the proximal end region and the distal end region and extending into the lumen; and a second flat region formed onto the side of the body at a second region between the proximal end region and the distal end region and extending into the lumen.

The proximal end region may be configured to receive an inversion support catheter having an expandable funnel on a distal end of the inversion support catheter. For example, the proximal region may have a larger diameter opening into the lumen of the introducer device than the distal end. In some variations the proximal end is flared open.

Any of these device may include a stop projecting from an outer surface of the distal end region of the body, wherein the stop is configured to limit the depth that the device is insertable into the sheath. The stop may be a ring. In some variations the stop does not extend completely around the outer surface of the distal end region, but includes a plurality of buttons or extensions extending at approximately the same longitudinal position.

The first flat region may be separated from the second flat region along a distal to proximal axis of the body. Alternatively or additionally, the first flat region and the second flat region may be opposite from each other at approximately the same (or overlapping) longitudinal position along the distal to proximal axis of the body. Any of these devices may include a third flat region formed on the side of the body opposite the first flat region and extending into the lumen. Any of these device may include a fourth flat region formed on the side of the body between the proximal end region and the distal end region and extending into the lumen.

The first flat region, the second flat region, or both the first and second flat regions may be angled relative to a distal to proximal axis of the body. For example, the walls on either side of the first and second flat region may be at an angle of between 20 degrees and 70 degrees relative to the distal to proximal axis of the body.

In general, these devices may be removable be tearing away. For example, any of these devices may include a tear line comprises a line of perforations extending down the length of the body (e.g., proximal to distal axis) either partially or completely. The tear line may comprise a line formed in the body having a thinner wall thickness than the rest of the body. In some variations the tear line is a frangible region configured to be pulled or broken.

Also described herein are systems including any of these introducers. For example, a system may include: an inversion support comprising a catheter and an expandable funnel at a distal end of the catheter; and an introducer, comprising: an elongate body having a lumen extending from a distal end to a proximal end, the body configured to tear along a defined tear line extending proximally to distally, wherein the lumen has a narrower diameter on at a proximal end region of the body than at a distal end region of the body; a first flat region formed on a side of the body between the proximal end region and the distal end region and extending into the lumen; and a second flat region formed on the side of the body between the proximal end region and the distal end region and extending into the lumen.

The system may also include a tractor comprising a flexible tube that extends distally in an un-inverted configuration along an outer surface of the catheter, inverts over a distal end opening of the expandable funnel, and extends proximally within a lumen of the catheter in an inverted configuration, wherein the flexible tube is configured to invert by rolling over the distal end opening when a first end of the tractor is pulled proximally within the catheter lumen.

Also described herein are methods of loading an inversion support having a catheter and an expandable funnel on a distal end of the catheter, e.g., using any of these devices (introducers). For example, a method may include: inserting the funnel into a distal end of an introducer; driving the funnel and the distal end of the catheter distally into the introducer; and collapsing the twisting the funnel against a first flat region formed on a side of the introducer body and a second flat region formed on the side of the introducer body as the funnel and catheter are driven distally into the introducer. The method may also include coupling the distal end of the introducer to a sheath.

In general, coupling may comprise inserting the introducer into the sheath until a stop on the introducer engages with the sheath. Collapsing and twisting may comprise driving the funnel against an angled flattened region of the first and/or second flat region.

Any of these methods may include removing the introducer by tearing along a tear line extending in a distal-to-proximal length of the introducer.

As mentioned above, also described herein are apparatuses (e.g., devices, systems, etc.) that have increase tractor lengths. In particular, these tractors may be woven tractors, and in particular, may be knitted tractors. The tractor may be packed onto the outer surface of the inversion support (e.g., the catheter of the inversion support). In some variations packing may result in compressing the tractor; this compression may be loose (e.g., so that it does not self-expand and does not need to be constrained against self-expanding) or loaded (e.g., so that it is constrained from self-expanding). Any of these apparatuses and methods may also include long sheaths that may allow delivery of a compressed weave into any sheath. The packed tractors described herein may pack a greater length of tractor into a smaller length of the device, reducing the need for supplemental or additional tractors to be loaded and used. The packing may increase the length of the extended tractor by up to 100% or more (typically between 30% and 90%, greater than 20%, greater than 30%, greater than 40%, greater than 50%, greater than 60%, etc.). The additional length may provide several advantages of a delivery a packed tractor, including better tracking through tortuosity and longer clot ingestion capacity.

For example, a system for removing a material from a vessel amy include: an inversion support comprising a catheter, the catheter having a distal end opening; a tractor comprising a flexible tube that extends distally in an un-inverted configuration along an outer surface of the catheter, inverts over the distal end opening and extends proximally within the catheter lumen in an inverted configuration, wherein the flexible tube is configured to invert by rolling over the distal end opening when a first end of the tractor is pulled proximally within the catheter lumen; and a stop on the outer surface of the catheter, wherein the stop prevents the tractor from extending proximally down the outer surface of the catheter without inhibiting the tractor from moving distally down the outer surface of the catheter, so that the stop maintains the un-inverted tractor on the outer surface of the catheter in a packed configuration that extends distally over the outer surface of the catheter as the tractor is pulled proximally within the catheter lumen.

In some variations a system for removing a material from a vessel may include: an inversion support comprising a catheter, the catheter having a distal end opening; a tractor comprising a flexible knitted tube that extends distally in an un-inverted configuration along an outer surface of the catheter, inverts over the distal end opening and extends proximally within the catheter lumen in an inverted configuration, wherein the flexible tube is configured to invert by rolling over the distal end opening when a first end of the tractor is pulled proximally within the catheter lumen; and a stop on the outer surface of the catheter, wherein the stop prevents the tractor from extending proximally down the outer surface of the catheter without inhibiting the tractor from moving distally down the outer surface of the catheter, further wherein the un-inverted tractor on the outer surface of the catheter is held in a packed configuration by the stop in which longitudinally adjacent loops of the knitted tube overlap by 40% or more along on the outer surface of the catheter in the proximal to distal direction, so that the tractor extends distally over the outer surface of the catheter as the tractor is pulled proximally within the catheter lumen.

The tractor, and particularly the knitted tractors, may be packed so that the knitted loops of the tractor overlap. In some variations the un-inverted tractor on the outer surface of the catheter is held in a packed configuration by the stop in which longitudinally adjacent loops of the knitted tube overlap by more than 40% (e.g., 45% or more, 50% or more, 55% or more, 60% or more, 65% or more, 70% or more, 75% or more, 80% or more, 85% or more, 90% or more, etc.) along on the outer surface of the catheter in the proximal to distal direction. For example, the adjacent loops of the knitted tube may overlap by more than 60% along on the outer surface of the catheter in the proximal to distal direction.

In some variations the packing of the tractor may be performed, reducing the longitudinal length of the tractor in the storage or pre-deployed configuration of the apparatus, without putting a substantial compressive force on the tractor, e.g., so that there is little if any internal force urging the tractor to self-expand. Thus, the stacking may still maintain the tractor in a “loose” configuration in which it is expanded by pulling, e.g., from the distal end, rather than self-expanding longitudinally. This may prevent the tractor from locking or jamming. In some variations this may be done by allowing the longitudinally adjacent loops to stack up onto each other, as described in detail herein. Alternatively, in some variations the tractor may be longitudinally packed so that there is a compressive force applied against an inherent self-expansion force when the tractor is packed. The force driving self-expansion may be adjusted by the adjusting the amount of compression and/or the type of tractor (e.g. number of loops per circumferential turn, shape-setting the tractor, material forming the tractor, thickness of the material forming the tractor weave/knit, etc.).

Any of these tractors may include a collar on a second end or end region of the tractor (e.g., the end outside of the inversion support catheter) that is configured to abut the stop so that the tractor is held in the packed configuration. The stop may comprise an annular ring.

In some variations the apparatus may also include an introducer covering the tractor, wherein the introducer comprises an introducer stop within a lumen of the introducer preventing the tractor from extending proximally.

For example, a system for reloading an inverting tube apparatus may include: an elongate puller having a distal end region and a proximal end region; a tractor comprising a flexible knitted tube that is coupled to a distal end region of the puller, wherein the tractor extends proximally from the distal end region in an un-inverted configuration along an outer surface of the puller; an introducer covering the tractor, wherein the introducer comprises an introducer stop within a lumen of the introducer preventing the tractor from extending proximally beyond the stop without inhibiting the tractor from moving distally, and a defined tear line extending proximally to distally configured to tear away to remove the introducer, wherein the un-inverted tractor on the outer surface of the puller is held in a packed configuration by the stop, wherein longitudinally adjacent loops of the knitted tube overlap by 40% or more along in the proximal to distal direction, further wherein the introducer is configured to receive an inversion support catheter between the tractor and the puller.

Also described herein are methods and devices for assisting in the manual operation of any of the apparatuses described herein. For example, described herein are grips, and systems including grips, that may alternately grip, release, and slide (and in some cases reset their position) on the inversion support, in a manner that allows the inversion support and tractor (via manipulation of a puller) to be more easily operated by a single user, e.g., using two hands.

For example, described herein are methods of removing material from a vessel using an inverting tractor apparatus, wherein the inverting tractor apparatus comprises an inversion support catheter having an inner lumen, a flexible tractor extending over an outer surface of the inversion support catheter, a compressible grip slideably disposed on a proximal end region of the outer surface of the inversion support catheter, and a puller within the lumen of the inversion support catheter, wherein a first end of the tractor is coupled to the puller, the method comprising: applying a pressure with a first hand to compress the grip onto the inversion support catheter and moving the compressed grip and the inversion support catheter distally into a sheath enclosing at least part of the inverting tractor apparatus so that a distal end region of the inversion support catheter is moved distally in the vessel; sliding the grip proximally over the inversion support catheter; pulling the puller relative to the inversion support catheter with a second hand concurrently with moving the inversion support catheter axially, and/or concurrently with moving the grip over the inversion support catheter, whereby the tractor is inverted over the catheter to draw the material into the lumen of the inversion support catheter.

In some variations the method of removing material from a vessel may include: advancing an inverting tractor apparatus through the vessel until a distal end portion of the apparatus is located proximate to the material, wherein the inverting tractor apparatus comprises an inversion support catheter having an inner lumen, a flexible tractor extending over an outer surface of the inversion support catheter, a compressible grip slideably disposed on a proximal end region of the outer surface of the inversion support catheter, and a puller within the lumen of the inversion support catheter, wherein a first end of the tractor is coupled to the puller; applying pressure with a first hand to compress the grip onto the proximal end region of the inversion support catheter; moving the compressed grip with the first hand to move the inversion support catheter axially relative to a sheath enclosing at least part of the inverting tractor apparatus; releasing the compression of the grip and moving the grip over the inversion support catheter with the first hand; and pulling the puller relative to the inversion support catheter with a second hand concurrently with moving the inversion support catheter axially, and/or concurrently with moving the grip over the inversion support catheter, whereby the tractor is inverted over the catheter to draw the material into the lumen of the inversion support catheter.

Sliding the grip may comprise manually sliding the grip proximally over the inversion support catheter using the first hand. In some variations, sliding the grip comprises automatically sliding the grip proximally using a bias after releasing the pressure from the first hand.

The grip may be finger operated. For example, applying the pressure with the first hand may comprise applying pressure to two side of the grip (e.g., between two or more fingers of the first hand) to clamp onto the inversion support catheter when pressure is applied. Applying the pressure may comprise applying pressure to two side of an elongate grip having a c-shaped profile.

Any of these methods may include repeating the steps of applying the pressure and sliding the grip while concurrently pulling the puller.

Also described herein are systems for removing a material from a vessel. For example, a system may include: an inversion support catheter having a lumen, a distal end region, a proximal end region, and a distal end opening; a puller with the lumen of the inversion support catheter; a tractor comprising a flexible tube that is attached to the puller at a first end, wherein the tractor extends distally in an un-inverted configuration along an outer surface of the inversion support catheter, further wherein the flexible tube is configured to invert by rolling over the distal end opening when the first end of the tractor is pulled proximally within the catheter lumen; and a compressible grip slideably coupled to the outer surface of the inversion support catheter.

The compressible grip may be configured to clamp onto the outer surface of the inversion support catheter when compressed between two fingers. In some variations the compressible grip comprises a flexible C-shaped cuff. In some variations the compressible grip comprises a pair of plates separated by a compressible material configured to clamp onto the outer surface of the inversion support catheter when compressed. Any of these compressible grips may include a silicone material (e.g., a silicone tube).

In general, the sheath may be configured to receive at least a portion of the inversion support catheter, tractor and puller. The sheath may be part of the system or separate from the systems (apparatuses) described herein. Any of these sheathes may include a hub.

Any of the grips described herein may be configured to automatically resume a predetermined position, relative to the sheath. For example, the grip may be configured with a bias (e.g., spring, etc.) having a set distance from the sheath. For example, the compressible grip may be coupled to the sheath with a biased return configured to return the compressible grip to an initial distance from the sheath when it is slid axially over the inversion support catheter.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A and 1B illustrate one example of an inverting tube apparatus for removing material from a body lumen, as described herein. FIG. 1A shows the apparatus (including an inversion support a flexible tractor tube, and a puller) having an expandable funnel at the distal end with the funnel in a collapsed configuration within an outer (e.g., delivery) cannula. FIG. 1B shows the apparatus with the funnel in an expanded configuration.

FIGS. 2A and 2B illustrate the operation of an inverting tube apparatus to capture material (e.g., clot) within a body lumen (e.g., blood vessel).

FIGS. 3A-3B illustrate alternative attempts to load an apparatus such as the one shown in FIGS. 1A-1B or 2A-2B into a sheath (e.g., sheath hub) for deploying into the body.

FIG. 4A shows one example of an introducer (e.g., funnel introducer) for introducing an inverting tube apparatus having an expandable funnel into a body, e.g., into a sheath.

FIG. 4B illustrates a mandrel onto which an introducer such as the one shown in FIG. 4A may be formed.

FIG. 4C illustrate a side view of an introducer similar to the one shown in FIG. 4A.

FIGS. 5A-5B illustrate one example of a method of using an introducer such as the one shown in FIGS. 4A-4C.

FIGS. 6A-6B show an example of a distal end of an introducer such as the one shown in FIGS. 4A-4C and 5A-5B including a stop limiting the depth into which the introducer may be inserted into the hub.

FIG. 7A shows an example of a portion of a tractor (e.g., a knitted tractor) extending over the outer surface of an inversion support (e.g., inversion support catheter) in an unstacked, e.g., uncompressed, configuration.

FIG. 7B shows an example of a portion of a tractor, such as the one shown in FIG. 7A, extending over the outer surface of an inversion support in a stacked (and in some configurations, compressed) configuration.

FIGS. 8A-8C illustrate examples of a tractor in unstacked (FIG. 8A) and stacked (FIGS. 8B and 8C) configurations, showing an approximately 50% compression in the axial (e.g., longitudinal) length of the tractor.

FIGS. 9A-9B illustrate an enlarged view of a knitted tractor in an unstacked (FIG. 9A) and stacked (FIG. 9B) configuration.

FIG. 10A illustrates one example of an apparatus in which the tractor is unstacked.

FIG. 10B shows an apparatus similar to that shown in FIG. 10A with the tractor in a stacked configuration, showing approximately 40% compression, and including a stop or lock on the outer surface of the catheter.

FIGS. 11A-11C illustrate one example of a stacked tractor including an introducer configured to be loaded (e.g., preloaded) into a sheath. FIG. 11A shows a stacked tractor within an elongate (e.g., tear-away) introducer with a second introducer, e.g., an inversion support introducer included therein. FIG. 11B illustrates the loading of an inversion support into the preloaded stacked tractor of FIG. 11A. FIG. 11C illustrates the pre-loaded apparatus, including the stacked tractor and inversion support, within the elongate introducer.

FIG. 12 is one example of a system including an inversion support and a pair of tractors each coupled to a puller.

FIG. 13 is another example of a system including an inversion support and a pair of tractors.

FIG. 14A illustrates a first example of a system including a grip that is configured for easy two-handed operation.

FIG. 14B is one example of a grip similar to the one shown in FIG. 14A.

FIG. 15A is another example of an inverting tube apparatus including a grip (similar to that shown in FIG. 14A) for operation by a single user, using two hands.

FIGS. 15B-15C illustrate operation of the apparatus of FIG. 15A.

FIG. 16A illustrates another example of an inverting tube apparatus including a grip.

FIGS. 16B-16C show a section through the grip portion of the apparatus of FIG. 16A, illustrating operation of the grip.

FIG. 17A illustrates another example of an inverting tube apparatus including a grip.

FIGS. 17B-17C show a section through the grip portion of the apparatus of FIG. 17A, illustrating operation of the grip.

FIGS. 18A-18C illustrate the operation of another example of an apparatus including a grip. In this example, the grip is coupled to the sheath with a biased return (e.g., spring) configured to return the grip to an initial distance from the sheath when it slides axially over the inversion support.

FIG. 19A illustrates one example of a length support that may be used with any of the apparatuses described herein to hold and organize the inversion support.

FIG. 19B illustrates the operation of the length support shown in FIG. 19A.

### DETAILED DESCRIPTION

In general, described herein are inverting tube apparatuses for removing material form a lumen of a subject's body and methods of using them. In particular, described herein are methods and apparatus for loading any of these apparatuses into a body, including introducers. Also described herein are method and apparatuses for removing larger and/or longer materials from the lumen of a vessel, including stacked tractor regions. Also included herein are methods and apparatuses for improving the insertion of these inverting tube apparatuses into the body.

Any of the inverting tube apparatuses described herein may include an inverting tractor that is configured to roll into an inversion support catheter and capture material from within a vessel.

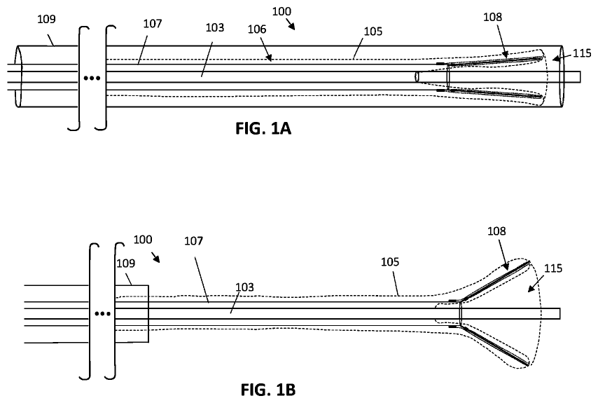

For example, FIG. 1A shows one variation of an apparatus 100, including an elongate inversion support 106. In this example, the elongate inversion support includes a catheter 107 having a distal end region that includes a distal end opening 115. In some variations (as shown here) the inversion support may include an expandable funnel 108 forming the distal end region of the inversion support. Alternatively, in some variations no funnel is included. In any of these inversion supports, the distal end region may have an increasing softness (measured by durometer, e.g., shore durometer) except that the very distal-most end region (distal end 115, including the distal end opening) may be substantially less soft than the region immediately proximate to it. Thus, although the distal tip region of the catheter (e.g., the distal most x linear dimensions, where x is 10 cm, 7 cm, 5 cm, 4 cm, 3 cm, 2 cm, 1 cm, 9 mm, 8 mm, 7 mm, 6 mm, 5 mm, 4 mm, 3 mm) has an increasing softness/decreasing harness extending from the proximal to distal ends, the very distal end region (e.g., measured as distal most z linear dimensions, where z is 1 cm, 9 mm, 8 mm, 7 mm, 6 mm, 5 mm, 4 mm, 3 mm, 2 mm, 1 mm, 0.8 mm, 0.5 mm, 0.3 mm, 0.2 mm, etc., and z is always at least three times less than x) has a hardness that is greater than the hardness of the region immediately proximal to it, and may be as hard or harder than the proximal-most region of the distal tip region.

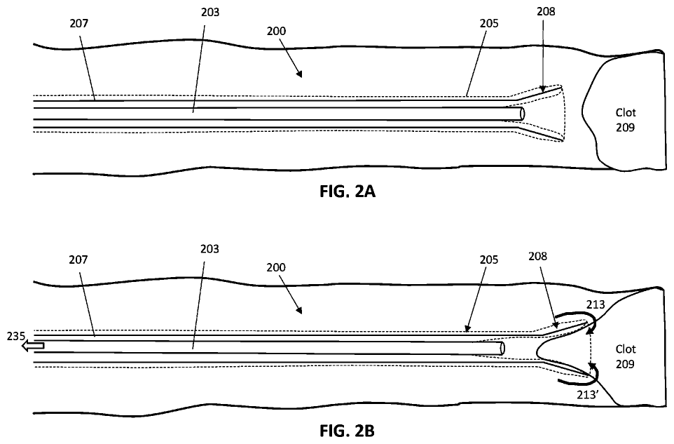

The apparatus also includes a tractor 105, comprising a flexible tube that extends over the outer surface of the inversion support, and is configured to invert over the distal end opening of the inversion support (e.g., funnel 108 in FIG. 1A), and extend in an inverted configuration within the inversion support in the proximal direction. The tractor may be coupled to an elongate puller 103, as shown in FIG. 1A. Pulling the tractor (e.g., by pulling the puller) proximally may invert the tractor over the distal end opening and into the inversion support. This may capture and pull material, as will be described in FIGS. 2A-2B, below. In some variations the tractor is a woven material. For example, in some variations the tractor is a knitted material. The knitted material may be, e.g., a knitted filament (e.g., wire) of Nitinol, stainless steel, polymer, etc. The tractor may include a number of loops extending helically around the circumference of the tractor tube.

In the example shown in FIG. 1A, the tractor, puller and inversions support are held initially within a catheter 109, configured as a delivery catheter here. In some variations the apparatus may be delivered to the target region from which material is to be removed using a delivery catheter. Either the tractor, puller and inversion support may be driven through the delivery catheter and/or the delivery catheter may be preloaded with the tractor, puller and inversion support and delivered to the target location. Any of these apparatuses may include or be configured for use with a guidewire (not shown), which may be passed through a guidewire lumen, e.g., within the puller.

As mentioned, the inversion support shown in FIG. 1A include an expandable funnel 108, shown collapsed/compressed in FIG. 1A, within the delivery catheter 109. FIG. 1B shows the apparatus of FIG. 1A deployed out of the delivery catheter 109 (e.g., extended distally out of the distal end of the delivery catheter). In this example, the funnel 108 is expanded.

Any of the elongate inversion supports described herein may include an elongate hollow catheter 107 having a column strength that is sufficient to prevent buckling when the catheter is pulled over the distal annulus (distal end opening). Thus, the elongate inversion support may be configured so that it does not collapse (e.g., buckle) when 500 g or less of compressive force is applied (e.g., at least about 700 g, 600 g, 500 g, 400 g, 300 g, etc. of compressive force) for neurovascular applications. For peripheral vascular applications the elongate inversion support may be selected or configured to withstand at least 1500 g of compressive force (e.g., at least about 2000 g, 1900 g, 1800 g, 1700 g, 1600 g, 1500 g, 1400 g, etc. of compressive force). Any of the apparatuses described herein may include an elongate inversion support that is not a full-length catheter, but may include a portion of a catheter, typically at the distal end, connected to a rod, wire, hypotube, or the like or may be skived. In some variations the distal end 115 of the elongate inversion support may be adapted so that the tractor 105 may slide or roll and invert over the distal end of the catheter without being caught (binding, jamming) or without substantial friction.

The tractor 105 may be attached to the puller 103 at or near the distal end or the puller (in some variations the tractor may be attached at a slightly proximal region of the puller, so that the puller distal end extends forward ahead of the puller when extended distally from the inversion support catheter. In this example the tractor 105 is configured to ride over the outer diameter of the inversion support so that it is within, e.g., about 1 mm or less (e.g., 0.5 mm or less) of the outer diameter of the inversion support catheter.

FIGS. 2A-2B show an apparatus 200 similar to that shown in FIGS. 1A-1B within a vessel of a body (e.g., a blood vessel) in which a clot 209 is located. The proximal end of the apparatus may be positioned adjacent to the clot. In some variations a delivery catheter (not shown) may be positioned within the vessel and the inversion support catheter and tractor (and in some variations puller) may be driven through the delivery catheter, e.g., over a guidewire, so that it is adjacent to the clot. FIG. 2A shows an example of a flexible tractor 205 coupled to a puller 203. In this example the tractor 205 is integrated with the puller 203, forming an assembly. In FIG. 2A, the tractor is a tube of material (e.g., knitted) that is flexible and elongate (longer than 20 cm). The tractor may have a relaxed inner diameter (in the un-inverted configuration) that is slightly greater than the outer diameter of the catheter of the elongate inversion support into which the tractor will be pulled; in the inverted configuration the inner diameter of the tractor may be between about 50% and 120% of the inner diameter of the inversion support catheter 207, which may prevent locking up of the tractor within the inversion support. The flexible and tubular tractor 205 may be sufficiently soft and flexible (e.g., having a low collapse strength) so as to easily roll and fold over the distal aperture of the elongate inversion support. The puller 203 may be, e.g., a hypotube. In some variations a puller is not necessary, but the internal end of the tractor 205 may extend proximally and be directly pulled.

In FIG. 2B, the tractor 205 is configured, e.g., by shape-setting (heat setting, etc.), to expand in the relaxed inverted configuration (when inverted within the inversion support catheter) to a radial diameter that is at least 60% of the inner diameter of the inversion support catheter (e.g., at least 65%, at least 70%, at least 75%, at least 80%, at least 85%, at least 90%, at least 95%, at least 100%, etc., e.g., between 0.7 and 4 times, between 0.8 and 4 times, between 0.7 and 2 times, etc.) when unconstrained. This may prevent the inverted tractor from collapsing down onto itself in the inverted configuration as mentioned above, even when pulled proximally into the catheter. This may also for the outward-flared shape as it inverts into the distal end opening of the catheter.

In operation, the apparatus may draw material (e.g., clot) into the inversion support. In FIG. 2B, the puller 203 is pulled proximally (arrow 235), while the inversion support is either held steady or advanced distally; this pulls the knitted tractor 205 over the distal end opening of the inversion support catheter so that it rolls 213, 213′ and inverts into the open funnel 208 of the inversion support catheter, drawing clot material with it.

Any of the tractors described herein may be, but are not limited to, knitted tractors. A knitted tractor may include rounded or more preferably teardrop-shaped loops each having a length per loop, 1, and a width per loop, w. The ratio of the length per loop to width per loop may be greater than 1 (e.g., the length may be longer than the width). For example, the length may be greater than twice the width, greater than 3× the width, greater than 4× the width, greater than 5× the width, greater than 6× the width, greater than 7× the width, greater than 8× the width, greater than 9× the width, etc. As will be described in greater detail below, the tractor may be stacked so that the tractor is in a stacked configuration in which laterally adjacent loops overlap by a percentage of the loop length (e.g., greater than 40% of the length, greater than 50% of the length, greater than 60% of the length, greater than 70% of the length, etc.).

In some variations of the inverting tube apparatuses (e.g., thrombectomy apparatuses) the knitted tractor may be shape set to have an inner diameter that is much larger than the outer diameter of the inversion support catheter. See, e.g., U.S. Patent Application Publication No. 2019/0336148. However, in some variations it may be desirable for the knitted tractor to have an inner diameter that is closer to the outer diameter of the catheter, particularly where narrower-profile apparatuses are desired. These apparatuses may also have a lower pulling friction (e.g., require a lower pulling force) due to reduced friction within the catheter.

In any of the apparatuses described herein, but in particular, apparatuses having a distally opening funnel, as shown in FIGS. 1A-1B and 2A-2B above, loading the apparatus into the body may be done using a sheath (e.g., hemostatic sheath and/or hemostatic valve) that is configured to receive the distal end of the device, e.g., into a sheath hub. However, it may be challenging to load the apparatus into a sheath where without jamming the apparatus and/or collapsing the distal end of the apparatus in a manner that may prevent its operation once delivered into the target region of the body.

For example, FIGS. 3A-3B illustrate techniques for inserting the distal end of an apparatus 300 into a sheath 301. In FIG. 3A, the apparatus includes the tractor 305, the puller 303 and the inversion support (including catheter 307 and expandable funnel 308 at the distal end). The funnel region may be pinched to be compressed for insertion, however, this may be difficult and may risk damaging the apparatus.

FIG. 3B shows another example of an apparatus 300′ being introduced into a sheath 301, in which the apparatus is included in an introducer 312 that may be configured to insert into the sheath. In this example, the introducer may coupled to the opening of the sheath hub and configured to allow the device to be driven distally into the body (e.g., within a delivery catheter, so that it can be deployed near the target tissue to be removed. Thus, the apparatus may be loaded into an introducer (e.g., a funnel introducer) that may then be loaded into the sheath, similar to that shown in FIG. 3B.

In general, an introducer, which may be referred to herein as a funnel introducer, may be configured to allow low-friction instruction of the apparatus with the collapsible/expandable funnel of the inversion support into a sheath. These introducers may avoid damaging the funnel and/or rest of the apparatus that may otherwise occur when pushing the apparatus (and/or the introducer holding the apparatus) into the sheath hub and/or when loading the apparatus into the introducer. For example, pushing the apparatus too hard/too far into the sheath hub may partially collapse the end of the introducer and circumferentially pinch and/or grab the funnel tip and make it hard to transfer funnel into the sheath. This problem may be particularly acute where the funnel is formed of a braided material (e.g., a braided funnel). See, e.g., U.S. patent application Ser. No. 16/594,256, filed Oct. 7, 2019, titled “INVERTING THROMBECTOMY APPARATUSES AND METHODS OF USE”, which is herein incorporated by reference in its entirety. In addition, if the introducer is not pushed far enough into the sheath hub, a dead space may be result, making it hard to transfer the funnel into the sheath.

In general, apparatuses including a funnel-shaped distal end (of the inversion support) may also be challenging to insert into an introducer and/or sheath because they require precise manipulation (e.g., by a user or technician) to rotate and control the collapsing of the funnel as it is inserted, without damaging or disordering the tractor. Although it may be done, this process may require practice and technical expertise to perform.

Described herein are introducers configured to address these issues. For example, the introducers described herein may be configured to control rotation of the funnel and/or tractor as it is inserted into the introducer and/or out of the introducer into the sheath in a manner that may be done quickly and without substantial training.

The introducers described herein may also be configured to allow users to easily load the inversion support including an expandable funnel into an introducer, prior to loading into sheath (for reload situations). These apparatuses may be configured so that loading the funnel tip into the introducer may automatically adjust the position of the funnel to collapse the funnel and prevent it from jamming in the introducer by including one or more channels or guides (e.g., flat regions) within the internal lumen of the introducer. This may allow the funnel to be loaded with minimal twisting of the funnel to load.

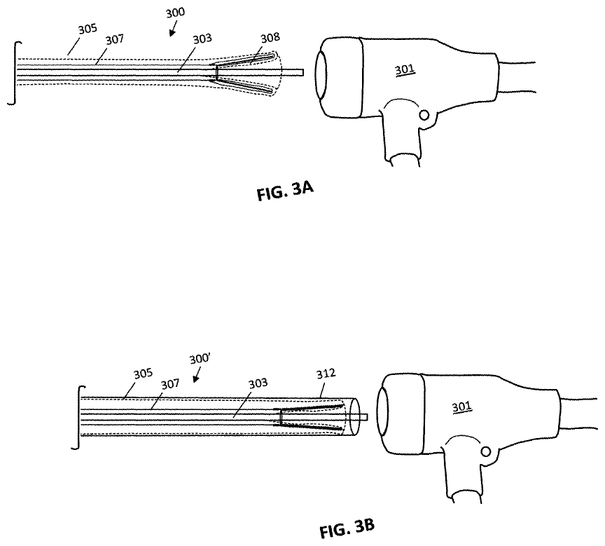

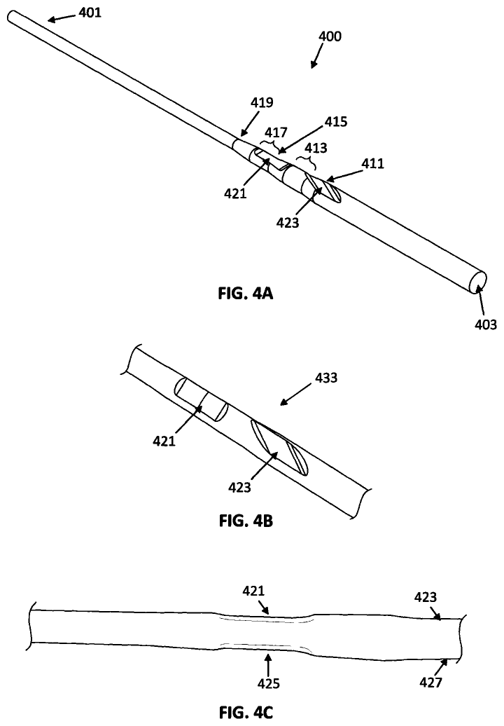

For example, FIG. 4A illustrates one example of a funnel introducer that may be used. In FIG. 4A the introducer has a distal end 401 and a proximal end 403; the distal end is narrower than the proximal end. In some variations the proximal end may be itself slightly funnel shaped to allow insertion of the expandable funnel. The funnel introducer may receive the apparatus (e.g., the inversion support including the expandable funnel, and the tractor) in the proximal end. The introducer includes a pair of flat regions 421, 423 along the length of the introducer, in a region of narrowing inner diameters. For example, in FIG. 4A, the first flat region 421 and the second flat region 423 are arranged along the distal length of the introducer. In some variations (as shown in FIG. 4C) opposite flat regions may be present on the opposite side of the introducer. The first and second flat regions may span a series of narrowing-diameter regions. The proximal region may have diameter 411 that tapers slightly down to a second diameter that is constant over a length 413 of the inserter, before again tapering down lightly to a slightly narrower diameter 415; this narrower diameter is sustained at a constant diameter over a second length 417 before again tapering down to a narrower diameter 419.

In FIG. 4A, either or both the first 421 and second 423 flat regions (and any complementary flat region on the opposite side of the introducer) may span a tapering region and a constant-diameter region, as shown.

These flat regions may be configured to help the collapsible funnel of the inversion support to twist (automatically) as it is loaded into introducer. In any of these examples the first and second flat regions may be present on one or both sides of the introducer. The second flat side 423 shown in FIG. 4A is angled relative to the long axis (e.g., the distal to proximal axis) of the introducer. The angle of the flat region relative to the long axis may be, for example, between about 10 degrees and about 80 degrees. Thus, the edges of the flat region of the first flat region 421 are perpendicular (approximately 90 degrees) relative to the long axis of the introducer (the proximal to distal axis), which the edges of the second flat region 423 are angled approximately 40 degrees relative to the long axis (e.g., between about 35 and 45 degrees). This angle of the flat region(s) may help spin the funnel so it collapses easily within the introducer.

FIG. 4B illustrates an example of a mandrel 433 over which an introducer such as the one shown in FIG. 4A may be formed. In FIG. 4B the flat regions 421, 423 are shown. FIG. 4C shows an example of a prototype inserter 400′ that has been configured as described herein, showing both the first flat region 421 and an opposite third flat region 425, as well as a portion of the second flat region 423 and an opposite fourth flat region 427.

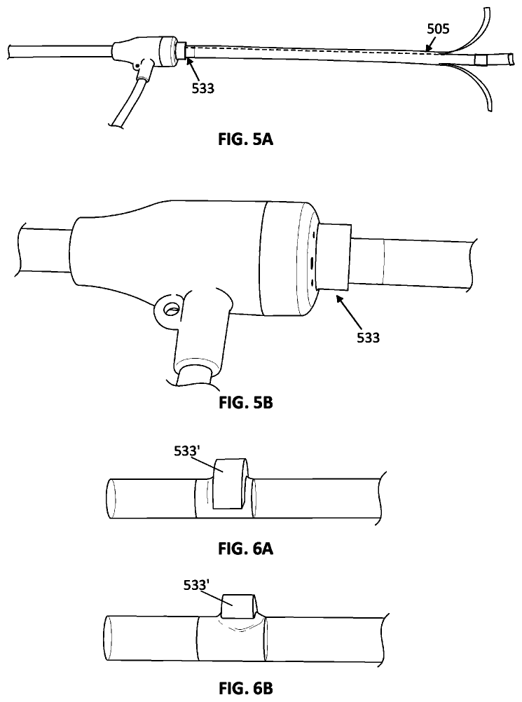

Any of these introducers may be peel-away introducers, as shown in FIG. 5A. Thus, any of these introducers may include one or more proximal-to-distal tear lines 505 along which the introducer may be torn to remove the introducer from over the apparatus as or after it has been introduced. The example in FIG. 5A also includes one or more stops 533. The stop may limit how far the introducer may be inserted into sheath hub. The stop may also be configured to be used with a peel away/tear away, so that the inserted may be torn or peeled through and/or past the stop. FIG. 5B shows an enlarged view of an introducer inserted into a sheath (sheath hub) to a stopping point set by the stop 533. FIGS. 6A-6B illustrate additional examples of stops 533′ that may be present at the distal end region of the introducer. In FIGS. 6A-6B, the stop is configured to project from just one side region of the introducer, and may allow the introducer to be a peel away (or “tear away”) introducer. The stop may be within about 0.5 mm (e.g., within about 0.8 mm, within about 1 mm, within about 1.5 mm, without about 2 mm, within about 3 mm, within about 3.5 mm, within about 4 mm, within about 5 mm, within about 7.5 mm, within about 10 mm, etc.) of the distal end of the introducer. The stop in FIGS. 6A-6B maybe a foamed material; other stop materials may be used.

### Stacked/Compressed Tractors

Also described herein are stacked tractors. Any of the inverting tube apparatuses described herein may be configured with a tractor that is stacked or compressed to have a higher density of tractor (e.g., knitted tractors having loops), per length. The apparatus may be configured to hold the stacked configuration of the tractor by including one or more holds, stops, and/or locks, e.g., on the inversion support and/or on an inserter for inserting into the body, etc. Stacking the tractor may be particularly effective when used with woven, including knitted, tractors. By maintaining a knitted tractor in a stacked configuration the amount of knit available to invert into the inversion support may be increased, and may also help with tracking and positioning of the apparatus, particularly in tortious anatomy, e.g., by making the effective weave length (e.g., loop length, 1) shorter for the same length of knitted tractor.

The stacked tractors described herein may be included with an assembled apparatus (e.g., loaded onto an inversion support and/or into a delivery catheter, or they may be included as a supplemental, reloading or replacement tractor that may be loaded into/onto an inversion support. Both features (the increased length of the tractor and the use of supplemental tractors) may increase the amount of material, such as clot, that may be removed.

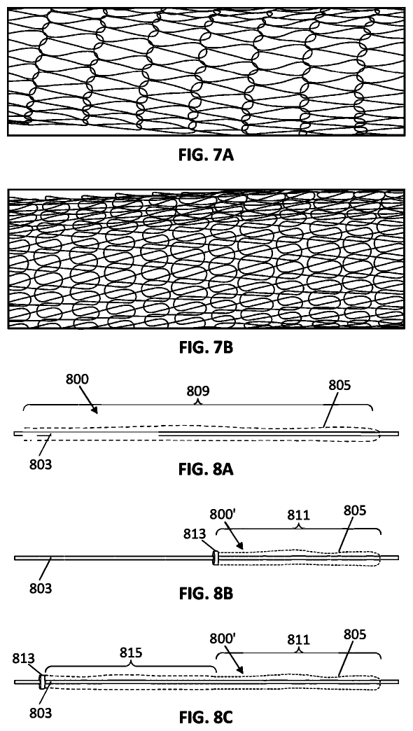

FIGS. 7A and 7B illustrate examples of a portion of an unstacked (e.g., non-stacked, FIG. 7A) knitted tractor, and a portion of a stacked (FIG. 7B) knitted tractor. In FIG. 7A the knitted tractor includes a plurality of loops, arranged in a helical pattern around the circumference of the tractor tube. Loops from adjacent rows may engage with each other, as shown. In the un-stacked configuration shown in FIG. 7A, although there is overlap between the loops that are longitudinally adjacent, this overlap is less than 10% of the total length (1) of each loop. Typically pulling one end of the tractor result in immediately moving the tractor in the direction that it is pulled. In contrast in FIG. 8 a similar tractor tube is shown in a stacked configuration in which the laterally adjacent loops overlap with each other over more than 20% of the length of each loop, thereby shortening the length of the tractor, or allowing a longer expanded tractor having as compared to an unstacked tractor having the same length as the stacked tractor. In FIG. 7B, the loops are more than 40% overlapping; in some variations the loops are more than 45%, more than 50%, more than 55%, more than 60%, more than 65%, more than 70%, more than 75%, more than 80%, more than 85%, more than 90%, etc. The thickness of the tractor over the stacked region may increase slightly as compared to an unstacked tractor having the same loop dimensions; however, because the loops may be formed of a filament (e.g., wire) that is relatively thin, the stacked thickness is still typically low (e.g., only about 0.1× to 2× as large as the unstacked thickness).

FIGS. 8A and 8B illustrate an example of a tractor assembly 800 including a tractor 805 coupled to a puller 803. The tractor 805 has a tractor length L_{unstacked} 809 in the unstacked configuration. The tractor tube material 805 may be woven tractor, such as but not limited to a knitted material. FIG. 8B shows a tractor that may have a similar knitted configuration, but is stacked. In FIG. 8B, the tractor assembly 800′ also includes the puller 803 and a tractor 805; the tractor is in a stacked configuration providing approximately 50% compression as compared to the unstacked configuration, given a length, L_{stacked}, 811. In this example the tracked tractor also includes a stack engagement region 813 at the proximal end of the tractor. The stack engagement region may operate as a stop of lock, or may function with a stop or lock, to prevent the tractor from unstacking proximally.

The stacked configuration may therefore permit a much longer tractor to be used for an equivalent total (stacked) length compared to the unstacked configuration. For example FIG. 8C shows an example of a stacked knitted tractor 800″ similar to FIG. 8B but with an additional length 815 of stacked tractor added to the proximal end, which may be secured in place via a stack engagement region 813.

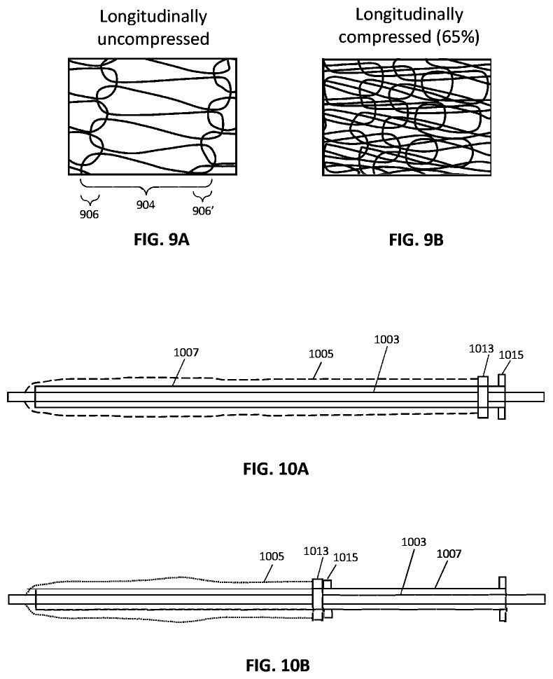

FIGS. 9A and 9B illustrate regions of knitted tractor in an unstacked (longitudinally uncompressed) configuration, shown in FIG. 9A and a stacked (longitudinally compressed) configuration, shown in FIG. 9B. The un-inverted tractor on the outer surface of the catheter may be packed into the packed configuration by increasing the amount of overlap between longitudinally adjacent loops, as shown between FIGS. 9A and 9B. In FIG. 9A there is relatively little overlap; each length, L 904, of the loop is overlapping 906, 906′ with less than 30% of its total length L. Longer loop lengths may allow for greater density of overlap and therefore greater stacking. In FIG. 9B, the entire loops overlap, as shown.

In any of these apparatuses, a stack engagement region may be included as part of the knitted tube. The stack engagement region may be a ring or loop (e.g., a loop of polymeric material) that may be held by a stop to prevent the tractor from unstacking in the proximal direction.

FIG. 10 shows an example of an apparatus including a tractor (show in an unstacked configuration) 1005, attached to a puller 1003, and extending over an inversion support 1007 (including an inversion support catheter). FIG. 10B shows the same apparatus with the tractor 1005 in a stacked configuration. The proximal end of the tractor includes a stack engagement region 1013 in both FIGS. 10A and 10B; the stack engagement region is configured as a loop or ring of material (e.g., a cuff that may include one or more cuts or perforations to allow it to invert when pulled over the distal end opening of the inversion support. In FIG. 10B, the stack engagement region 1013 is retained against a stop 1015 (also referred to as a stack stop or a stack retaining stop) on the inversion support. The stop 1015 may be a projection or a set of projections extending from, and/or around all or part of the circumference of the inversion support catheter. In some variation the stop is a ring. The stop may prevent the tractor tube from expanding proximally.

In use the stacked tractor may be pulled distally into the inversion support as described generally for tractors above; the tractor may be unstacked from the distal end preferentially as it is pulled, leaving the more proximal regions stacked. This may also beneficially prevent locking or jamming of the tractor onto the outer surface of the inversion support, may assist in tracking, and/or may reduce the force required to pull the tractor into the inversion support (e.g., and remove material).

In some variations a tractor may be configured as a replacement of additional tractor that may be used to reload an apparatus. These supplemental (e.g., reloading or replacement) tractors may be stacked as described herein. In addition, in some variations it may be beneficial to cover all or a portion of the tractor with a cover to prevent manual contact that may interfere with the tractor, e.g., stacking of the tractor and/or the configuration of the tractor over the outer surface of an inversion support, etc. In some variations it may be beneficial to avoid handling of the tractor as much as possible.

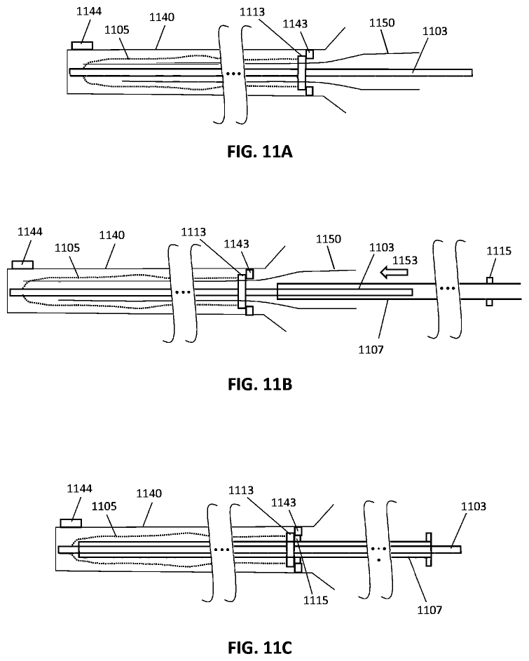

For example, any of the apparatuses (e.g., systems) described herein may include a cover, which may also be configured as an introducer, for covering all or a portion of a tractor in the stacked configuration. In some variations this cover (e.g. inserter) may also be configured to hold the tractor in a compressed configuration. For example, FIG. 11A illustrates one example of an apparatus configured as a replacement or secondary tractor that may be loaded onto/with an inversion support and/or inserted into a body lumen, e.g., through a sheath. In this example, the cover/introducer 1140 may be long, e.g., long enough to cover the majority of the length of the tractor; the tractor 1105 (shown as a stacked tractor) may be coupled at one end to the puller 1103 (e.g., at a distal end region of the puller) and may extend proximally over the puller. The tractor may be held in the stacked configuration by engaging a stack engagement region 1113 against a stop 1140 (e.g., a stack stop) that project into the lumen of the cover/introducer.

The cover/introducer may be a tear-away cover/introducer, and may include one or more tear lines extending down the distal-to-proximal length of the device (not shown). The cover/introducer 1140 may also include an insertion stop 1144 at the proximal end region to limit how far into the sheath the introducer may be inserted, as described above for the funnel introducer.

In some variations a second introducer or sleeve 1150 may be inserted between all or a portion of the tractor 1105 and the puller 1103; this second introducer or sleeve may help in loading the inversion support onto/into the tractor, as illustrated in FIG. 11B. In some variation the inversion support may be loaded without the need for a second introducer. The second introducer may be referred to as an inner introducer (or inner cheater) and may also be a peel-away introducer (e.g., having one or more tear away lines or regions that may extend partially or completely down the length of the inner introducer.

For example, in FIG. 11B, an inversion support 1107 is inserted 1153 over the puller 1103 and into the second introducer 1150, as shown. Once inserted partially or completely, the second introducer 1150 may be withdrawn and removed, e.g., by tearing it along its length to remove. The inversion support may also include one or more stops 1115 that may retain the tractor in the stacked configuration even after removal of the outer sleeve/inserter 1140. FIG. 11C illustrates an example of the loaded tractor after removing the second/inner introducer 1150 and advancing the inversion support fully distally so that the stop 1115 on the outer surface of the inversion support engages against the stack engagement region 1113. In this example, the long cover/introducer 1140 may then be coupled to a sheath (e.g., sheath hub) and inserted to a patient, as described above, peeling away the long cover/introducer 1140. In this manner, a second or additional (e.g., third, fourth, etc.) tractor may be inserted and used after an initial (first) tractor has been fully withdrawn. The inversion support may be re-used and may be reloaded on the tractor as described.

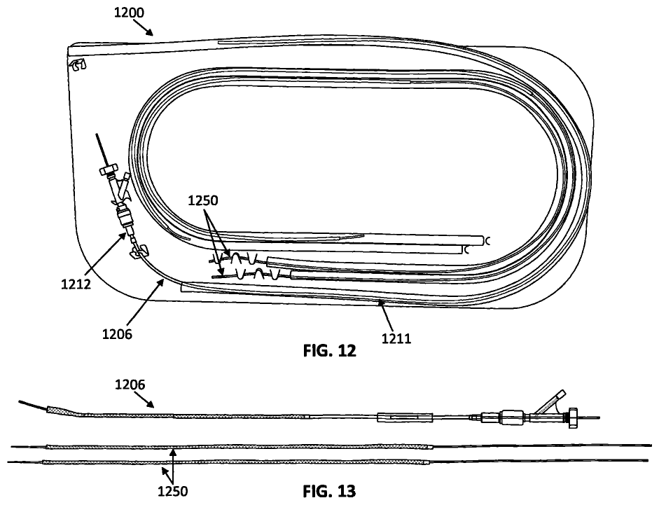

Thus any of the apparatuses described herein may include a system incorporating any of the features and elements described herein. For example, FIG. 12 illustrates one example of a system 1200 including a first (or initial) assembly 1206 that includes an inversion support (including an inversion support catheter), a puller and a tractor over the outside of the inversion support and coupled to the puller. The proximal end of the assembly may include a hub 1212 for controlling inversion support, a hemostat valve and/or the puller control. The system may also include one or more (two are shown) additional tractors 1250 and pullers that may be loaded with the inversion support catheter and re-inserted as described above. Any of these tractors may be stacked tractors. Further, any of the introducers described above may be used or included with these systems.

FIG. 13 shows the assembly 1206 of FIG. 12, and the two additional tractors and pullers 1250, outside of the covers shown in FIG. 12. In FIG. 13 the tractors are held in the stacked configuration.

GRIP

Also described herein are apparatuses including a finger grip that is configured to slide over the outside of a distal end region of the inversion support catheter, and to grip onto (and prevent sliding) when gripping pressure is applied. These grips may allow the apparatuses described herein to be easily manipulated and handled by a single user using both hands. The user's first hand may be used to operate the grip for moving the inversion support distally and/or proximally, while the user's second hand may be used to manipulate the puller to pull the puller proximally, rolling the tractor into the device to capture material. This may allow the inversion support to be pushed or pulled relative to a sheath (for inserting the device into the body of the subject), while using the other hand to operate the puller.

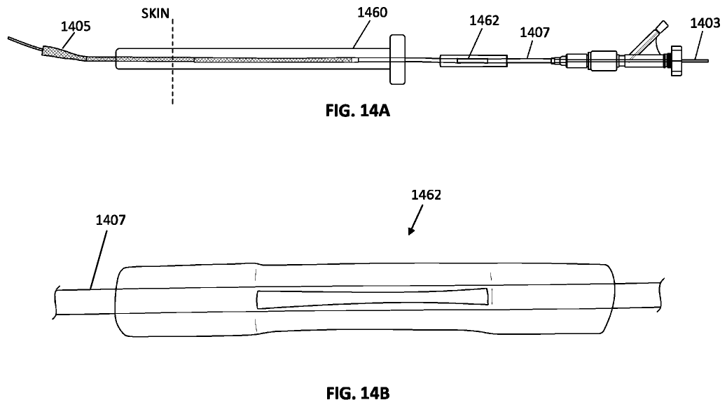

FIG. 14A illustrates this operation. In FIG. 14A an apparatus 1400 including an inversion support 1407, a puller 1403 and a tractor 1405 coupled to the puller at a first and extending over the outer surface of the inversion support, so that it may be pulled into the lumen of the inversion support as the inversion support is static or pushed distally, to invert and grab material. In FIG. 14A, the apparatus is inserted into the body through a guide sheath and/or guide catheter 1460.

FIG. 14B shows an enlarged view of the grip 1462 on the outer surface of the inversion support 1407. In this embodiment the grip is a silicone pusher that is a tube having a larger inner diameter (ID) than the outer diameter (OD) of the inversion support catheter (e.g., by greater than 10%, 15%, 20%, 25%, 30%, etc.), so that it may ride/slide easily over the outer surface of the inversion support catheter, until pressure is applied to squeeze it and grip the outer surface of the inversion support catheter. The outer diameter of the grip (“gripper”) may be oversized, to permit it to be more easily grabbed. For example, the outer dimeter may be greater than 2× the outer diameter of the inversion support catheter (e.g., greater than 2.5×, greater than 3×, etc.).

In FIG. 14B the grip is configured as a tube having one or more longitudinal slots cut into the middle region of the tube (e.g., in the proximal-to-distal axis). In this configuration, the slots allow the grip to be more easily compressed by applying finger pressure.

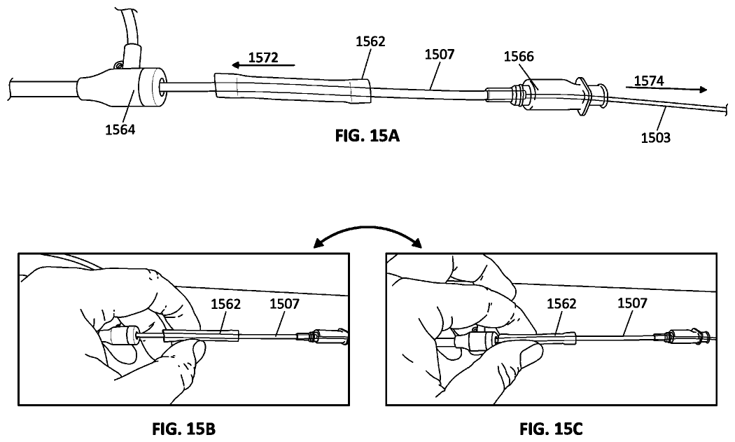

FIG. 15 shows another example of an apparatus similar to that shown in FIG. 14A and 14B, including a gripper 1562 that rides over the distal end region of the inversion support 1507. In FIG. 15 the apparatus is inserted into the sheath (sheath hub 1564), and the grip resides between the sheath hub and the proximal end of the inversion support 1566. The inversion support 1507 may be advanced by squeezing the grip and pushing it closer to the sheath 1564, as indicated by the arrow 1572. This may be done with a single hand (the user's first hand). The user may concurrently pull or otherwise manipulate the puller 1503 extending from the distal end (hub) of the inversion support. As shown in FIGS. 15B and 15C, this process may be repeated and reciprocated to advance and roll the tractor to capture material from within the body lumen (e.g., vessel); as shown in FIG. 15B, the grip may be squeezed to engage with the outside of the inversion support, then pushed distally (into the sheath hub). After advancing, the pressure applied by the fingers may be relaxed, as shown in FIG. 15C, and the grip slid back, proximally, (e.g., 1-2 inches) and the process repeated (FIG. 15B). As mentioned, the puller may be pulled concurrently to roll the tractor and invert it into the inversion support.

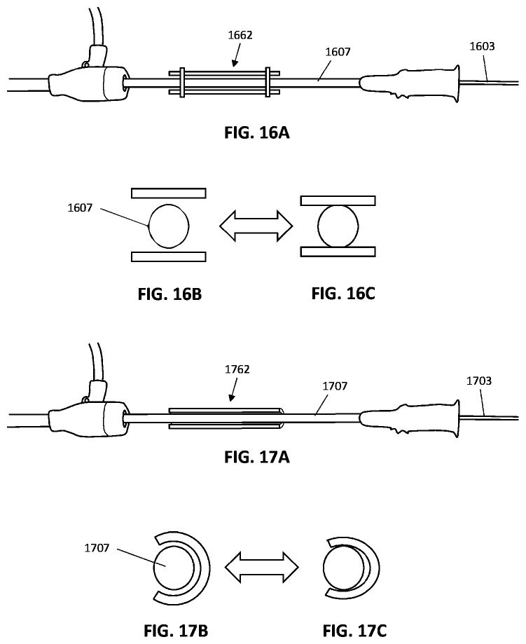

Any appropriate grip may be used. For example, FIGS. 16A-16C illustrate another example of a grip 1662, configured as a pair of elongate surfaces (which may include a silicone or other material) separated by a compressible material (e.g., foam, elastic, etc.). FIGS. 16B and 16C illustrate the grip in the un-engaged (e.g., not squeezed, free to slide over the inversion support 1607), and engaged (e.g., squeezed, gripping onto the inversion support 1607). The user's second hand may manipulate the puller 1603.

FIGS. 17A-17C illustrate another example of a grip 1762, configured as a C-shaped tube, e.g., of silicone. FIGS. 17B and 17C illustrate the grip in the un-engaged (e.g., not squeezed, free to slide over the inversion support 1707), and engaged (e.g., squeezed, gripping onto the inversion support 1707). The user's second hand may manipulate the puller 1703. This embodiment may be applied/removed direction through the lateral opening, and may therefore be beneficial.

Alternatively, in some variations the user may also or alternatively wear gloves that are selectively sticky or gripping for one or more region (e.g., the outer surface of the inversion support catheter, and/or the puller.

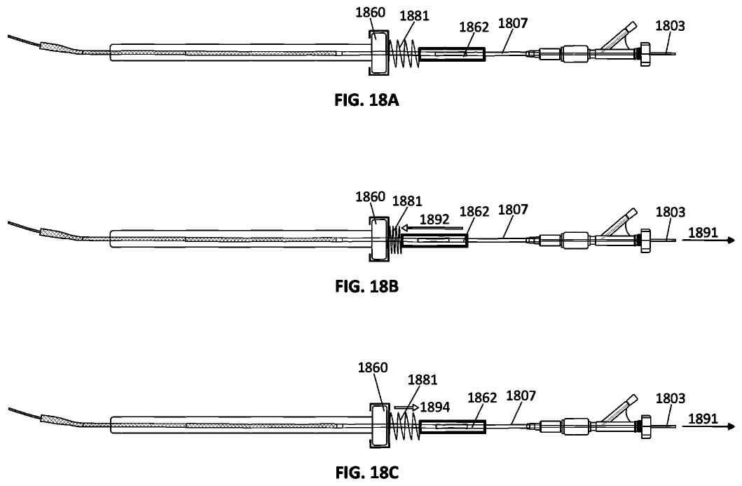

Any of the grips described herein may be configured to automatically return to a pre-set position during operation, e.g., when the gripping force is released. This may simplify operation. For example, in some variations a bias (e.g., spring, elastic material, etc.) may be connected between the grip and the hub of the sheath, as shown in FIGS. 18A-18C. In this example, the sheath hub 1860 may couple to the return bias 1881 that is also coupled to the grip 1862. FIG. 18A shows the apparatus in a first position in which the grip 1862 is in a set “neutral” position on the outer surface of the inversion support 1807, as held by the return bias 1881. The user's first hand may hold the grip 1862 between two fingers and may hold onto the hub (e.g., with the palm or other portions of the first hand. The puller 1803 may be gripped by a second hand.

In FIG. 18B, at a second position, the user may squeeze the grip 1862 and pull 1892 the grip and therefore the inversion support 1807 proximally, compressing the bias 1881 relative to the sheath hub 1860, as shown. Concurrently, the user may pull 1891 the puller 1803 proximally. Thereafter, the user may release the force applied to pinch the grip 1862 onto the inversion support 1807, allowing it to return to the neural position as the bias 1881 applies a return force 1894, as shown in FIG. 18C. Thus, in FIG. 18C, the grip is returned to the first position. In some variations the inversion support catheter may also be pulled proximally (stretching the bias 1881), by pinching or squeezing the grip and sliding it and the inversion support proximally (not shown); the grip will similarly return to the neutral position after release.

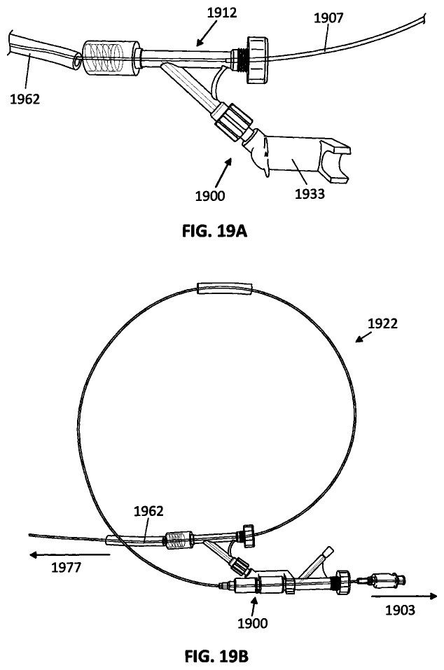

Any of these apparatuses may also include one or more loop control clasps, as shown in FIGS. 19A and 19B. For example, FIG. 19A shows a loop control clasp 1900 that may be included as part of any of these systems that is configured to attach to the inversion support 1907 (e.g., a hub on the inversion support 1912) and includes a clip 1933 for securing another portion of the inversion support catheter, as shown in FIG. 19B. This may allow the inversion support to form a loop which may be easier for a user to control. This may improve ergonomics, particularly where a clot may more proximally located and therefore leaving a longer length of the apparatus outside of the body. In some variations the clasp may be integrated into the hub 1912; alternatively the clasp 1900 may be configured to couple to the hub 1912 or to another region of the inversion support.

FIG. 19B illustrates the use of the clasp shown in FIG. 19A. In use, the clasp may secure a more distal end of the inversion support catheter to form a loop 1922. The device may then be operated as described above, including advancing the inversion support distally (e.g., by pushing a finger grip 1962 and inversion support length distally 1977; the puller 1903 (coupled to the tractor) may be pulled proximally at the same time.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the disclosed inventions. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the disclosed inventions.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the inventions as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the inventions as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A device for introducing an inversion support catheter into a delivery sheath, the device comprising:
an elongate body having a lumen extending from a distal end region to a proximal end region, the body configured to tear along a defined tear line extending proximally to distally, the lumen having a first diameter at the proximal end region of the body and a second diameter at the distal end region of the body, wherein the second diameter is greater than the first diameter,
wherein a sidewall of the body defining the lumen comprises a first flat region and a second flat region spaced apart from the first flat region along a distal to proximal axis of the body.

2. The device of claim 1, wherein the proximal end region is configured to receive an inversion support catheter having an expandable funnel disposed on a distal end of the inversion support catheter.

3. The device of claim 1, further comprising a stop projecting from an outer surface of the distal end region of the body, wherein the stop is configured to limit a depth that the device is insertable into the delivery sheath.

4. The device of claim 1, wherein the sidewall of the body defining the lumen further comprises a third flat region circumferentially spaced apart from, so as to be opposing, the first flat region.

5. The device of claim 4, wherein the sidewall of the body defining the lumen further comprises a fourth flat region circumferentially spaced apart from, so as to be opposing, the second flat region.

6. The device of claim 1, wherein one or both of the first flat region and the second flat region is angled relative to the distal to proximal axis of the body.

7. The device of claim 1, wherein the tear line comprises a line of perforations.

8. The device of claim 1, wherein the tear line comprises a linear region formed in the body having a thinner wall thickness than the rest of the body.

9. A method of loading an inversion support into a sheath, the inversion support comprising a catheter and an expandable funnel disposed on a distal end of the catheter, the method comprising:
inserting the funnel into an open distal end of an introducer;
advancing the respective funnel and the distal end of the catheter distally into the introducer; and
collapsing and twisting the funnel against first and second flat regions formed on an interior surface of the introducer as the funnel and catheter are advanced distally into the introducer.

10. The method of claim 9, further comprising coupling the distal end of the introducer to the sheath.

11. The method of claim 10, wherein coupling the distal end of the introducer to the sheath comprises inserting the introducer into the sheath until a stop on an exterior surface of the introducer engages with the sheath.

12. The method of claim 9, wherein collapsing and twisting the funnel comprises advancing the funnel against a respective angled flattened portion of the first flat region and/or second flat region.

13. The method of claim 9, wherein collapsing and twisting the funnel comprises advancing the funnel against a respective angled flattened portion of each the first flat region and second flat region.

14. The method of claim 9, further comprising uncoupling the introducer from the sheath by tearing along a tear line extending a distal-to-proximal length of the introducer.

15. A system for removing a material from a body vessel, the system comprising:
an inversion support comprising a catheter, the catheter having a lumen and a distal end opening in communication with the lumen;
a tractor comprising a flexible tube having an un-inverted portion that extends distally along an outer surface of the catheter, and wherein the flexible tube inverts over the distal end opening of the catheter, wherein an inverted portion of the flexible tube extends proximally within the catheter lumen in an inverted configuration, wherein the flexible tube is configured to invert by rolling over the distal end opening when a first end of the tractor is pulled proximally within the catheter lumen; and
a stop on the outer surface of the catheter, wherein the stop prevents the tractor from moving proximally along the outer surface of the catheter without inhibiting the tractor from moving distally along the outer surface of the catheter, wherein the stop maintains the un-inverted portion of the tractor on the outer surface of the catheter in a packed configuration as the tractor is pulled proximally within the catheter lumen.

16. The system of claim 15, wherein the tractor is a knitted tube.

17. The system of claim 16, wherein, when the un-inverted portion of the knitted tube on the outer surface of the catheter is held in a packed configuration, longitudinally adjacent loops of the knitted tube overlap by more than 40% along on the outer surface of the catheter.

18. The system of claim 17, wherein the longitudinally adjacent loops of the knitted tube overlap by more than 60% along on the outer surface of the catheter.

19. The system of claim 15, further comprising a collar on a second end of the tractor, wherein the collar is configured to abut the stop such that the tractor is held in the packed configuration.

20. The system of claim 15, further comprising an introducer covering the tractor, wherein the introducer comprises an introducer stop within a lumen of the introducer, wherein the introducer stop prevents the tractor from moving proximally along the outer surface of the catheter.
